(19) United States
(12) Patent Application Publication
(10) Pub. No.: US 2026/0083732 A1
(43) Pub. Date: Mar. 26, 2026

(54) UPADACITINIB FORMULATION

(71) Applicant: INTAS PHARMACEUTICALS LTD., Ahmedabad, Gujarat (IN)

(72) Inventors: Rahul Sudhakar PATIL, Ahmedabad (IN); Sandip Kumar Manubhai PATEL, Ahmedabad (IN); Ashutosh JAMLOKI, Ahmedabad (IN); Ashish SEHGAL, Ahmedabad (IN)

(21) Appl. No.: 19/109,053

(22) PCT Filed: Sep. 6, 2023
(86) PCT No.: PCT/IB2023/058794

§ 371 Date: Mar. 5, 2025

(30) Foreign Application Priority Data
Sep. 9, 2022 (IN) 202221051592

(51) Int. Cl. A61K 31/4985; A61K 9/20; A61K 9/28

(52) U.S. Cl. CPC A61K 31/4985; A61K 9/2009; A61K 9/2013; A61K 9/2018; A61K 9/2054; A61K 9/2095; A61K 9/2893

(57) ABSTRACT

The present invention is related to pharmaceutical composition of upadacitinib comprising upadacitinib or its pharmaceutically acceptable salt thereof, release controlling polymer, pH modifier and one or more pharmaceutical acceptable excipient; wherein the release controlling polymer is hydrophobic polymer.

### RELATED APPLICATIONS

This application is related to Indian Provisional Application No. IN202221051592 filed on 9 Sep. 2022 and is incorporated herein in its entirety.

### FIELD OF THE INVENTION

The present invention is related to a pharmaceutical composition of upadacitinib comprising upadacitinib or its pharmaceutically acceptable salt thereof, release control polymer, pH modifier and one or more pharmaceutically acceptable excipients, wherein the said release control polymer is hydrophobic polymer.

### BACKGROUND OF THE INVENTION

Upadacitinib is a Janus kinase (JAK) inhibitor and is chemically described as (3S,4R)-3-Ethyl-4-(3H-imidazo[1,2-a]pyrrolo[2,3-e]pyrazin-8-yl)-N-(2,2,2-trifluoroethyl) pyrrolidine-1-carboxamide hydrate (2:1).

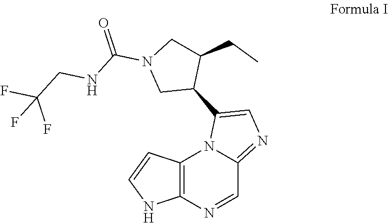 [SMILES: CC[C@@H]1CN(C(=O)NCC(F)(F)F)C[C@@H]1c1cnc2cnc3[nH]ccc3n12]

Upadacitinib is approved in the form of extended-release tablets and marketed by Abbvie under the brand name RINVOQ® (Innovator product).

The extended-release tablets are approved in the strengths of 15 mg, 30 mg and 45 mg and are indicated for the treatment of rheumatoid arthritis, psoriatic arthritis, atopic dermatitis, ulcerative colitis and ankylosing spondylitis.

U.S. Pat. No. 8,426,411 patent discloses upadacitinib or pharmaceutically acceptable salts, stereoisomers and isomers thereof. The compounds of the invention are useful for treating immunological and oncological conditions. U.S. Pat. No. 8,962,629 discloses various compound including upadacitinib or a pharmaceutically acceptable salts, pro-drugs, biologically active metabolites, stereoisomers, and isomers thereof.

U.S. Pat. Nos. 10,344,036, 9,963,459, 10,202,394 and 1,020,239 patents disclose an extended-release solid dosage form comprising upadacitinib or pharmaceutically acceptable salt thereof, an acidic pH modifier and a release control polymer; wherein the release control polymer is a hydrophilic polymer, and the acidic pH modifier is an organic acid, wherein upadacitinib is also in crystalline hemihydrate form.

U.S. Pat. Nos. 10,519,164 and 10,730,883 patents discloses an solid oral extended release once-daily pharmaceutical composition comprising upadacitinib free base equivalent, wherein the solid oral extended release once-daily pharmaceutical composition is bioequivalent to an oral extended release once-daily film-coated tablet consisting of: (i) the intragranular core having freebase hydrate form C, microcrystalline cellulose, and hydroxypropyl methyl cellulose; (ii) the extragranular core having microcrystalline cellulose, mannitol, tartaric acid, hydroxypropyl methyl cellulose, colloidal silicon dioxide/silica, and magnesium stearate; and (iii) film coat comprising polyvinyl alcohol, titanium dioxide, polyethylene glycol 3350, talc, and iron oxide.

U.S. Pat. No. 11,198,697 patent discloses an extended-release tablet for oral administration comprising upadacitinib, at least one filler, at least one a release control polymer and at least one additional pharmaceutically acceptable excipient selected from the group consisting of one or more pH modifiers, one or more glidants, one or more surfactants and one or more lubricants.

U.S. Pat. No. 9,879,018 patent discloses a pharmaceutical composition comprising an amorphous freebase of upadacitinib and a pharmaceutically acceptable carrier.

WO2022147073 patent application discloses an extended-release solid dosage form comprising upadacitinib, or a pharmaceutically acceptable salt thereof, at least one pH dependent polymer and at least one release control material, wherein the pH dependent polymer is hydroxypropylmethyl cellulose acetate succinate (HPMCAS) or alginic acid, wherein the at least one release control polymer is selected from the group consisting of hydroxypropylmethyl cellulose (HPMC), hydroxypropyl cellulose, hydroxyethyl cellulose, a copolymer of acrylic acid crosslinked with a polyalkenyl polyether (Carbopol), non-ionic homopolymer of ethylene oxide (Polyox), a water soluble natural gum of a polysaccharide, crosslinked starch, polyvinyl acetate, polyvinylpyrrolidone, and combinations thereof.

The above prior-art references disclose that the marketed compositions comprising upadacitinib are having several disadvantages; for example, as storage time increases without appropriate moisture protection, the tablet appearance grows increasingly mottled, decrease in dissolution rate and increased levels of impurity profile. This may be attributed to the presence of relatively high amount of Tartaric acid and Hydrophilic polymer which makes the formulation more sensitive to moisture uptake if not stored appropriately. A formulation that improves the moisture uptake can alleviate this disadvantage of marketed formulation. A suitable approach to decrease the moisture uptake can be incorporation of hydrophobic excipients in formulation while maintaining all other quality attributes in the acceptable domain. Thus, development of a hydrophobic matrix formulation presents an attractive alternate. The present invention provides a pharmaceutical composition of upadacitinib comprising upadacitinib or its pharmaceutically acceptable salt thereof, release control polymer, pH modifier and one or more pharmaceutical acceptable excipients, wherein the said release control polymer is hydrophobic polymer and has comparable dissolution properties equivalent to commercialized upadacitinib tablet dosage form.

### OBJECT OF THE INVENTION

The primary object of the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer.

Another object of the present invention is to provide a pharmaceutical composition of upadacitinib comprising upadacitinib or its pharmaceutically acceptable salt thereof, release control polymer, pH modifier and one or more pharmaceutical acceptable excipient, wherein upadacitinib is present in an amorphous form.

Another object of the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and upadacitinib is present in an amorphous form.

Another object of the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer selected from the group consisting of glyceryl dibehenate, ethyl cellulose and combinations thereof.

Another object of the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and wherein the said pH modifier is selected from the group consisting of tartaric acid, fumaric acid, citric acid, succinic acid, malic acid, and combinations thereof.

Another object of the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and wherein the said one or more pharmaceutical acceptable excipient is selected from one or more diluents, one or more solvents, one or more glidants, one or more lubricants or the mixtures thereof.

Another object of the present invention is to provide a process for preparation of pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer.

Another object of the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and wherein release of upadacitinib is in line with the innovator product when the said composition is subjected to the dissolution test using USP Type 1 apparatus method at a rotation of 100 rpm in 0.05 M Phosphate Buffer pH 6.8 in 900 mL of volume.

Another object of the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and wherein release of upadacitinib is not less than 60% when measure at 8 hours in a dissolution test using USP Type 1 apparatus method at a rotation of 100 rpm in 0.05 M Phosphate Buffer pH 6.8 in 900 mL of volume.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer; upadacitinib is present in an amorphous form; and wherein release of upadacitinib is in line with the innovator product when the said composition is subjected to the dissolution test using USP Type 1 apparatus method at a rotation of 100 rpm in 0.05 M Phosphate Buffer pH 6.8 in 900 mL of volume.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer; upadacitinib is present in an amorphous form; and wherein release of upadacitinib is not less than 60% when measure at 8 hours in a dissolution test using USP Type 1 apparatus method at a rotation of 100 rpm in 0.05 M Phosphate Buffer pH 6.8 in 900 mL of volume.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; upadacitinib is present in an amorphous form; the said pH modifier is selected from the group consisting of tartaric acid, fumaric acid, citric acid, succinic acid, malic acid, and combinations thereof; and wherein release of upadacitinib is in line with the innovator product when the said composition is subjected to the dissolution test using USP Type 1 apparatus method at a rotation of 100 rpm in 0.05 M Phosphate Buffer pH 6.8 in 900 mL of volume.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein the said composition does not have more than 1.0% (w/w) of total impurity of upadacitinib, after being stored at specific storage conditions.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein administration of a pharmaceutical composition of upadacitinib to healthy subjects under fasting conditions results in a mean Cmax from about 85 ng/ml to about 125 ng/ml of upadacitinib.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein administration of a pharmaceutical composition of upadacitinib to healthy subjects under fed conditions results in a mean Cmax from about 85 ng/mL to about 125 ng/ml of upadacitinib.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein administration of a pharmaceutical composition of upadacitinib to healthy subjects under fasting conditions results in a mean AUC from about 80 ng·h/mL to about 120 ng·h/mL of upadacitinib.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein administration of a pharmaceutical composition of upadacitinib to healthy subjects under fed conditions results in a mean AUC from about 80 ng·h/mL to about 95 ng·h/mL of upadacitinib.

Another object of the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein the said composition is bioequivalent to innovator product Rinvoq® under both fasting and fed conditions.

### SUMMARY OF THE INVENTION

The present invention related to a pharmaceutical composition of upadacitinib comprising upadacitinib or its pharmaceutically acceptable salt thereof, release control polymer, pH modifier and one or more pharmaceutical acceptable excipients, wherein the said release control polymer is hydrophobic polymer.

### DETAILED DESCRIPTION

Unless otherwise indicated, terms in this specification are intended to have their ordinary meaning in the relevant art.

The present invention is related to a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient, wherein upadacitinib is present in an amorphous form.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and upadacitinib is present in an amorphous form.

The term “Upadacitinib” used throughout the specification refers to not only their base per se, but also their other pharmaceutically acceptable salt, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof.

The term “pharmaceutically acceptable” means salt, carriers, excipients, and other formulation ingredients that are compatible with all other pharmaceutical ingredients of a composition and are not deleterious to an individual treated with composition.

In accordance with the present invention, the term “about” shall mean a variation up to 10% (plus or minus 10%) of the particular term.

The release control polymer is a hydrophobic polymer. Specifically, the said hydrophobic polymer is selected from a group consisting of glyceryl dibehenate, ethyl cellulose or mixtures thereof. The hydrophobic polymer can be present in a concentration of from about 40% to about 50% by weight of the total weight of the composition.

The pH modifier can be selected from the group consisting of tartaric acid, fumaric acid, citric acid, succinic acid, malic acid or mixtures thereof, more preferably tartaric acid. The pH modifier can be present in a concentration of from about 20% to about 35% by weight of the total weight of the composition.

In one of the embodiments, present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer selected from the group consisting of glyceryl dibehenate, ethyl cellulose and combinations thereof.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and wherein the said pH modifier is selected from the group consisting of tartaric acid, fumaric acid, citric acid, succinic acid, malic acid, and combinations thereof.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and wherein the said one or more pharmaceutical acceptable excipient is selected from one or more diluents, one or more solvents, one or more glidants, one or more lubricants or the mixtures thereof.

The diluents can be selected from the group comprising of but not limited to mannitol, dibasic calcium phosphate anhydrous, microcrystalline cellulose, corn starch, sucrose or other sugar or sugar derivatives, low substituted HPC, pregelatinized starch or mixture thereof, more preferably microcrystalline cellulose and mannitol. The diluents can be present in a concentration of from about 16% to about 40% by weight of the total weight of the composition.

The glidant can be selected from the group comprising of colloidal silicon dioxide, magnesium silicate, starch, talc, tribasic calcium phosphate, polyethylene glycol, carnauba wax or mixtures thereof, more preferably colloidal silicon dioxide. The glidant can be present in a concentration of from about 0.25% to about 1% by weight of the total weight of the composition.

The lubricant can be selected form the group comprising of agar, calcium stearate, ethyl oleate, ethyl laureate, glycerin, glyceryl palmitostearate, hydrogenated vegetable oil, magnesium oxide, magnesium stearate, sodium stearyl, sorbitol, stearic acid, talc, and zinc stearate or mixture thereof, more preferably magnesium stearate. The lubricant can be present in a concentration of about 0.5% to about 4% by weight of the total weight of the composition.

The solvent can be selected from the group of pharmaceutically acceptable solvents comprising of purified water, dichloromethane, isopropyl alcohol or mixtures thereof.

In one of the embodiments, the present invention is to provide a process for preparation of pharmaceutical composition of upadacitinib comprising upadacitinib or its pharmaceutically acceptable salt thereof, release control polymer, pH modifier and one or more pharmaceutical acceptable excipients; wherein the said release control polymer is hydrophobic polymer. Further, one of the process for preparation of pharmaceutical composition of upadacitinib according to the present invention comprising following steps:
1) Co-sifting Upadacitinib with pH modifier and diluent and mixing it to obtain a blend.
2) The blend of step 1 is granulated with required quantity of purified water.
3) The wet granules are wet milled through Co-mill and are dried in Rapid dryer.
4) The dried granules of step 3 are passed through Co-mill with suitable screen.
5) The extragranular materials i.e., release control polymer and glidant are co-sifted and blended with sized granules.
6) Lubricant is added and lubricated the blend of step 5.
7) The Lubricated blend of step 6 is compressed into tablets using compression machine with suitable punches.
8) The core tablets are coated with coating agent using auto coater.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipients; wherein the said release control polymer is hydrophobic polymer and wherein release of upadacitinib is in line with the innovator product when the said composition is subjected to the dissolution test using USP Type 1 apparatus method at a rotation of 100 rpm in 0.05 M Phosphate Buffer pH 6.8 in 900 mL of volume.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient, wherein the said release control polymer is hydrophobic polymer and wherein release of upadacitinib is not less than 60% when measure at 8 hours in a dissolution test using USP Type 1 apparatus method at a rotation of 100 rpm in 0.05 M Phosphate Buffer pH 6.8 in 900 mL of volume.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer; upadacitinib is present in an amorphous form; and wherein release of upadacitinib is in line with the innovator product when the said composition is subjected to the dissolution test using USP Type 1 apparatus method at a rotation of 100 rpm in 0.05 M Phosphate Buffer pH 6.8 in 900 mL of volume.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer; upadacitinib is present in an amorphous form; and wherein release of upadacitinib is not less than 60% when measure at 8 hours in a dissolution test using USP Type 1 apparatus method at a rotation of 100 rpm in 0.05 M Phosphate Buffer pH 6.8 in 900 mL of volume.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; upadacitinib is present in an amorphous form; the said pH modifier is selected from the group consisting of tartaric acid, fumaric acid, citric acid, succinic acid, malic acid, and combinations thereof; and wherein release of upadacitinib is in line with the innovator product when the said composition is subjected to the dissolution test using USP Type 1 apparatus method at a rotation of 100 rpm in 0.05 M Phosphate Buffer pH 6.8 in 900 mL of volume.

The term “stable” as used throughout the specification, refers to a pharmaceutical composition in which the active pharmaceutical ingredient upadacitinib is present in an amount of at least 90% of the original label specified amount for each such ingredient during specific storage conditions.

The term “specific storage conditions” as used throughout the specification, refers to the pharmaceutical composition of present invention stored for at least 6 months at 40° C./75% RH and 25° C./60% RH.

The term “AUC0-t” as used throughout the specification, refers to the steady state area under the plasma concentration time curve from time zero to the time of the last measured concentration calculated using the trapezoidal rule.

The term “Cmax” as used throughout the specification, refers to the plasma concentration of upadacitinib at Tmax, expressed herein as ng/mL, produced by the oral ingestion of a single dose, or indicated number of doses, of the dosage form or pharmaceutical composition, such as the dosage forms and compositions of the present disclosure. Unless specifically indicated, Cmax refers to the overall maximum observed concentration.

The term “Tmax” as used throughout the specification, refers to the time to peak plasma concentration of upadacitinib after oral ingestion of a single dose, or indicated number of doses of upadacitinib.

In accordance with the present invention, an assay value of upadacitinib in pharmaceutical composition of upadacitinib is within the limits of 90% to 110% after stability study according to ICH guidelines which is comparable when compared with reference product Rinvoq®.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein the said composition does not have more than 1.0% (w/w) of total impurity of upadacitinib, after being stored at specific storage conditions.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein administration of a pharmaceutical composition of upadacitinib to healthy subjects under fasting conditions results in a mean Cmax from about 85 ng/mL to about 125 ng/ml of upadacitinib.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof.; and wherein administration of a pharmaceutical composition of upadacitinib to healthy subjects under fed conditions results in a mean Cmax from about 85 ng/ml to about 125 ng/ml of upadacitinib.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof.; and wherein administration of a pharmaceutical composition of upadacitinib to healthy subjects under fasting conditions results in a mean AUC from about 80 ng·h/mL to about 120 ng·h/mL of upadacitinib.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and .wherein administration of a pharmaceutical composition of upadacitinib to healthy subjects under fed conditions results in a mean AUC from about 80 ng·h/mL to about 95 ng·h/mL of upadacitinib.

In one of the embodiments, the present invention is to provide a pharmaceutical composition of upadacitinib comprising upadacitinib or its pharmaceutically acceptable salt thereof, release control polymer, pH modifier and one or more pharmaceutical acceptable excipient; wherein the said release control polymer is hydrophobic polymer selected from the group consisting of glyceryl dibehenate, ethyl cellulose and combinations thereof; and wherein the said composition is bioequivalent to innovator product Rinvoq® under both fasting and fed conditions.

The present invention has been described by way of example only. It is to be recognized that modifications falling within the scope and spirit of the claims, which would be obvious to a person skilled in the art based upon the disclosure herein, are also considered to be included within the scope of this invention. The scope of the invention is in no manner limited by the disclosed example.

### Example 1

| Sr. No | Ingredients | % w/w |
|---|---|---|
| 1 | Upadacitinib | 1.0-10.0 |
| 2 | pH modifier | 20.0-35.0 |
| 3 | Diluent | 16.0-40.0 |
| 4 | Release Control Polymer | 40.0-50.0 |
| 5 | Glidant | 0.25-1.0 |
| 6 | Lubricant | 0.5-4.0 |
| 7 | Solvent | Qs |
| Core tablet weight | | 100 |
| 8 | Coating Agent | Qs |

| | |
|---|---|
| Coated tablet weight | — |

### Manufacturing Process:

1. Co-sifting Upadacitinib with pH modifier and diluent and mixing it to obtain a blend.
2. The blend of step 1 is granulated with required quantity of purified water.
3. The wet granules are wet milled through co-mill & wet milled granules are dried in Rapid dryer.
4. The dried granules of step 3 are passed through Co-mill with suitable screen.
5. The extragranular materials i.e., release control polymer and glidant are co-sifted and blended with sized granules of step 5.
6. Lubricant is added and lubricated the blend of step 5.
7. The Lubricated blend of step 6 is compressed into tablets using compression machine with suitable punches.
8. The core tablets are coated with coating agent using auto coater.

### Example 2

| | | |
|---|---|---|
| Sr. No | Ingredients | mg |

Intragranular materials

| | | |
|---|---|---|
| 1 | Upadacitinib | 15.0 |
| 2 | Tartaric Acid | 90.0-110.0 |
| 3 | Microcrystalline Cellulose | 85.0-95.0 |
| 4 | Mannitol | 90.0-100.0 |

Granulating Fluid

| | | |
|---|---|---|
| 5 | Purified water | Qs |

Extragranular Part

| | | |
|---|---|---|
| 6 | Microcrystalline Cellulose | 75.0-85.0 |
| 7 | Glyceryl Dibehenate | 65.0-75.0 |
| 8 | Colloidal Silicon Dioxide | 1.25-5.0 |

Lubrication

| | | |
|---|---|---|
| 9 | Magnesium Stearate | 5.0-10.0 |

| | |
|---|---|
| Core Tab weight (mg) | 460 |

### Manufacturing Process:

1. Upadacitinib, Tartaric acid, Microcrystalline Cellulose and Mannitol are co-sifted through ASTM #30 sieve and mixed.
2. Granulation: The blend of step 1 is granulated with required quantity of purified water.
3. The wet granules are wet milled through Co-mill.
4. The wet milled granules are dried in Rapid dryer at 60° C. to get LOD less than 2.5% w/w.
5. The dried granules of step 4 are passed through Co-mill with suitable screen.
6. The extragranular materials i.e. Glyceryl Dibehenate, Microcrystalline Cellulose and Colloidal Silicon Dioxide are co-sifted through ASTM #40 sieve and blended with sized granules of step 5.
7. Magnesium Stearate (sifted through ASTM #60) is added and lubricated with blend of step 6.
8. The Lubricated blend of step 7 is compressed into tablets using compression machine with suitable punches.

### Example 3

| | | |
|---|---|---|
| Sr. No | Ingredients | mg |

Intragranular materials

| | | |
|---|---|---|
| 1 | Upadacitinib | 30.0 |
| 2 | Tartaric Acid | 90.0-105.0 |
| 3 | Microcrystalline Cellulose | 60.0-70.0 |
| 4 | Mannitol | 70.0-80.0 |

Granulating Fluid

| | | |
|---|---|---|
| 5 | Purified water | Qs |

Extragranular Part

| | | |
|---|---|---|
| 6 | Microcrystalline Cellulose | 20.0-30.0 |
| 7 | Glyceryl Dibehenate | 80.0-100.0 |
| 8 | Ethyl cellulose | 80.0-100.0 |
| 9 | Colloidal Silicon Dioxide | 1.25-5.0 |

Lubrication

| | | |
|---|---|---|
| 10 | Magnesium Stearate | 5.0-15.0 |

| | |
|---|---|
| Core Tab weight (mg) | 480 |

### Manufacturing Process:

1. Upadacitinib, Tartaric acid, Microcrystalline Cellulose and Mannitol are co-sifted through ASTM #30 sieve and mixed.
2. Granulation: The blend of step 1 is granulated with required quantity of purified water.
3. The wet granules are wet milled through Co-mill.
4. The wet milled granules are dried in Rapid dryer at 60° C. to get LOD less than 2.5% w/w.
5. The dried granules of step 4 are passed through Co-mill with suitable screen.
6. The extragranular materials i.e. Glyceryl Dibehenate, Microcrystalline Cellulose, Ethyl cellulose and Colloidal Silicon Dioxide are co-sifted through ASTM #40 sieve and blended with sized granules of step 5.
7. Magnesium Stearate (sifted through ASTM #60) is added and lubricated with blend of step 6.
8. The Lubricated blend of step 7 is compressed into tablets using compression machine with suitable punches.
Example 4

| | | |
|---|---|---|
| Sr. No | Ingredients | mg |

Intragranular materials

| | | |
|---|---|---|
| 1 | Upadacitinib | 30.0 |
| 2 | Tartaric Acid | 100.0-125.0 |
| 3 | Microcrystalline Cellulose | 20.0-40.0 |
| 4 | Mannitol | 60.0-80.0 |

Granulating Fluid

| | | |
|---|---|---|
| 5 | Purified water | Qs |

Extragranular Part

| | | |
|---|---|---|
| 6 | Glyceryl Dibehenate | 100.0-125.0 |
| 7 | Ethyl cellulose | 100.0-125.0 |
| 8 | Colloidal Silicon Dioxide | 1.25-5.0 |

Lubrication

| | | |
|---|---|---|
| 9 | Stearic Acid | 10.0-20.0 |

| | |
|---|---|
| Core Tab weight (mg) | 500 |

Coating

| | | |
|---|---|---|
| 10 | Opadry | 15 |
| 11 | Purified water | Qs |

| | |
|---|---|
| Coated Tablet Weight (mg) | 515 |

### Manufacturing Process:

1. Upadacitinib, Tartaric acid, Microcrystalline Cellulose and Mannitol are co-sifted through ASTM #30 sieve and mixed.
2. Granulation: The blend of step 1 is granulated with required quantity of purified water.
3. The wet granules are wet milled through Co-mill.
4. The wet milled granules are dried in Rapid dryer at 60° C. to get LOD less than 2.5% w/w.
5. The dried granules of step 4 are passed through Co-mill with suitable screen.
6. The extragranular materials i.e. Glyceryl Dibehenate, Ethyl cellulose and Colloidal Silicon Dioxide are co-sifted through ASTM #40 sieve and blended with sized granules of step 5.
7. Stearic Acid (sifted through ASTM #60) is added and lubricated with blend of step 6.
8. The Lubricated blend of step 7 is compressed into tablets using compression machine with suitable punches.
9. The core tablets are coated with Opadry dispersion using auto coater.

### Example 5

| | | |
|---|---|---|
| Sr. No | Ingredients | mg |

Intragranular materials

| | | |
|---|---|---|
| 1 | Upadacitinib | 5.0-50.0 |
| 2 | Tartaric Acid | 100.0-175.0 |
| 3 | Microcrystalline Cellulose | 40.0-80.0 |
| 4 | Mannitol | 80.0-120.0 |

Granulating Fluid

| | | |
|---|---|---|
| 5 | Purified water | Qs |

Extragranular Part

| | | |
|---|---|---|
| 6 | Glyceryl Dibehenate | 75.0-125.0 |
| 7 | Ethyl cellulose | 50.0-100.0 |
| 8 | Colloidal Silicon Dioxide | 1.25-5.0 |

Lubrication

| | | |
|---|---|---|
| 9 | Magnesium Stearate | 5.0-10.0 |

| | |
|---|---|
| Core Tab weight (mg) | 500 |

Coating

| | | |
|---|---|---|
| 10 | Opadry | 15 |
| 11 | Purified water | Qs |

| | |
|---|---|
| Coated Tablet Weight (mg) | 515 |

### Manufacturing Process:

1. Upadacitinib, Tartaric acid, Microcrystalline Cellulose and Mannitol are co-sifted through ASTM #30 sieve and mixed.
2. Granulation: The blend of step 1 is granulated with required quantity of purified water.
3. The wet granules are wet milled through Co-mill.
4. The wet milled granules are dried in Rapid dryer at 60° C. to get LOD less than 2.5% w/w.
5. The dried granules of step 4 are passed through Co-mill with suitable screen.
6. The extragranular materials i.e. Glyceryl Dibehenate, Ethyl cellulose and Colloidal Silicon Dioxide are co-sifted through ASTM #40 sieve and blended with sized granules of step 5.
7. Magnesium Stearate (sifted through ASTM #60) is added and lubricated with blend of step 6.
8. The Lubricated blend of step 7 is compressed into tablets using compression machine with suitable punches.
9. The core tablets are coated with Opadry dispersion using auto coater.

### Dissolution Study

The composition of example 4 was tested for dissolution study as per USP Type 1 apparatus (Basket) at 100 rpm using 0.05 M Phosphate Buffer pH 6.8 in 900 mL of volume and gave the following results as Table 1.

TABLE 1

| Sr. | Time Points | Upadacitinib ER tablets | |
|---|---|---|---|
| No. | (Hrs) | 30 mg (RINVOQ ®) | 30 mg |
| 1 | 1 | 22 | 29 |
| 2 | 2 | 36 | 42 |
| 3 | 4 | 52 | 57 |
| 4 | 6 | 68 | 69 |
| 5 | 8 | 78 | 76 |
| 6 | 10 | 85 | 83 |
| 7 | 12 | 88 | 87 |
| 8 | 16 | 92 | 94 |
| 9 | 20 | 98 | 97 |

The composition of example 5 was tested for dissolution study as per USP Type apparatus 1 (basket) at 100 rpm using 0.05 M Phosphate Buffer pH 6.8 in 900 mL of volume and gave the following results as Table 2.

TABLE 2

| | Rinvoq ® | | Rinvoq ® | | Rinvoq ® | |
|---|---|---|---|---|---|---|
| | ER | Upadacitinib | ER | Upadacitinib | ER | Upadacitinib |
| Time | Tablet | ER Tablet | Tablet | ER Tablet | Tablet | ER Tablet |
| (Hrs) | 15 mg | 15 mg | 30 mg | 30 mg | 45 mg | 45 mg |
| 0.5 | 13 | 26 | 12 | 27 | 24 | 25 |
| 1 | 22 | 41 | 22 | 37 | 29 | 36 |
| 2 | 37 | 57 | 36 | 49 | 34 | 51 |
| 4 | 55 | 79 | 52 | 68 | 53 | 68 |
| 6 | 69 | 85 | 68 | 82 | 64 | 78 |
| 8 | 79 | 95 | 78 | 90 | 73 | 85 |
| 10 | 85 | 98 | 85 | 93 | 78 | 91 |
| 12 | 89 | 99 | 88 | 95 | 82 | 91 |
| 16 | 94 | 101 | 92 | 95 | 86 | 93 |

The above dissolution study shows the release of upadacitinib ER tablet is similar to the innovator product Rinvoq®.

The composition of example 5 was tested for dissolution study as per USP Apparatus 1 (basket) at 100 rpm using 0.1 N HCl in 900 mL of volume and gave the following results as Table 3.

TABLE 3

| | Upadacitinib ER | Upadacitinib ER | Upadacitinib ER |
|---|---|---|---|
| Time (Hrs) | Tablet 15 mg | Tablet 30 mg | Tablet 45 mg |
| 0.5 | 33 | 31 | 28 |
| 1 | 47 | 43 | 39 |
| 2 | 63 | 59 | 55 |
| 4 | 82 | 78 | 73 |
| 6 | 94 | 89 | 83 |
| 8 | 100 | 95 | 90 |
| 10 | 102 | 98 | 92 |
| 12 | 103 | 100 | 94 |
| 16 | 105 | 100 | 94 |

The composition of example 5 was tested for dissolution study as per USP Apparatus 1 (basket) at 100 rpm using Acetate Buffer pH 4.5 in 900 mL of volume and gave the following results as Table 4.

TABLE 4

| | Upadacitinib ER | Upadacitinib ER | Upadacitinib ER |
|---|---|---|---|
| Time (Hrs) | Tablet 15 mg | Tablet 30 mg | Tablet 45 mg |
| 0.5 | 30 | 27 | 27 |
| 1 | 41 | 38 | 39 |
| 2 | 56 | 53 | 54 |
| 4 | 75 | 71 | 71 |
| 6 | 87 | 82 | 81 |
| 8 | 94 | 89 | 87 |
| 10 | 97 | 93 | 90 |
| 12 | 99 | 94 | 92 |
| 16 | 101 | 96 | 93 |

### Stability Study:

TABLE 5
Stability data of Upadacitinib Extended Release Tablets 15 mg
(composition of example 5) under 6 month time interval at condition
of 40° C./75% RH and 25° C./60% RH in HDPE Bottle.

| | | | 40° C./ 75% RH | | 25° C./ 60% RH | |
|---|---|---|---|---|---|---|
| Test | Limit | Initial | 3 M | 6 M | 3 M | 6 M |
| Water | NMT 8.0% | 1.72% | 1.18% | 0.94% | 1.19% | 2.05% |
| determination | w/w | | | | | |
| (By KF) | | | | | | |
| Total impurity | NMT 1.0% | 0.03% | 0.03% | BQL | 0.03% | BQL |
| | w/w | | | | | |
| Assay | 90-110% | 101.40% | 100.50% | 100.90% | 101.00% | 101.00% |

TABLE 6
Stability data of Upadacitinib Extended Release Tablets 45 mg
(composition of example 5) under 6 month time interval at condition
of 40° C. / 75% RH and 25° C. / 60% RH in HDPE Bottle.

| | | | 40° C./ 75% RH | | 25° C./ 60% RH | |
|---|---|---|---|---|---|---|
| Test | Limit | Initial | 3 M | 6 M | 3 M | 6 M |
| Water | NMT 8.0% | 2.01% | 1.05% | 0.98% | 1.02% | 0.95% |
| determination | w/w | | | | | |
| (By KF) | | | | | | |
| Total impurity | NMT 1.0% | 0.015% | 0.019% | BQL | 0.019% | BQL |
| | w/w | | | | | |
| Assay | 90-110% | 98.70% | 99.40% | 99.70% | 99.10% | 98.00% |

The above data shows a total impurity not more than 1% in the formulation and assay value within the range of ICH guideline, indicative of stability of upadacitinib in the drug product.

### Bioequivalence Study:

Upadacitinib Extended-Release Tablets 45 and Rinvoq® ER Tablet 45 mg (composition of example 5) were subjected to bio-equivalence study under fasting and fed conditions and results are tabulated below.

TABLE 7

| | | % | |
|---|---|---|---|
| | Param- | Ratio | 90% |
| | eters | (T/R) | CI |
| Bioequivalence study under Fasting conditions | Cmax | 105.3 | 90.77- |
| Test | (ng/mL) | | 122.25 |
| Upadacitinib Extended-Release Tablets 45 mg | AUC0-t | 99.7 | 85.82- |
| Vs. | (ng · | | 115.31 |
| Rinvoq ® ER Tablet 45 | h/mL) | | |
| Bioequivalence study under Fed conditions | Cmax | 101.9 | 87.29- |
| Test | (ng/mL) | | 119.0 |
| Upadacitinib Extended-Release Tablets 45 mg | AUC0-t | 86.9 | 81.76- |
| Vs. | (ng · | | 92.45 |
| Rinvoq ® ER Tablet 45 | h/mL) | | |

The outcome of bioequivalence studies shows that pharmaceutical composition according to the present invention is bioequivalent to the Rinvoq® under both fasting and fed conditions.

We claims:

1. A pharmaceutical composition of upadacitinib comprising upadacitinib or its pharmaceutically acceptable salt thereof, release control polymer, pH modifier and one or more pharmaceutical acceptable excipients; wherein the said release control polymer is hydrophobic polymer.

2. The pharmaceutical composition of upadacitinib according to claim 1, wherein the said upadacitinib is present in an amorphous form.

3. The pharmaceutical composition of upadacitinib according to claim 1, wherein the said hydrophobic polymer is selected from the group consisting of glyceryl dibehenate, ethyl cellulose and combinations thereof.

4. The pharmaceutical composition of upadacitinib according to claim 1, wherein the said pH modifier is selected from the group consisting of tartaric acid, fumaric acid, citric acid, succinic acid, malic acid, and combinations thereof.

5. The pharmaceutical composition of upadacitinib according to claim 1, wherein the said one or more pharmaceutical acceptable excipient is selected from one or more diluents, one or more solvents, one or more glidants, one or more lubricants or the mixtures thereof.

6. A process for preparation of pharmaceutical composition of upadacitinib comprising upadacitinib or its pharmaceutically acceptable salt thereof, release control polymer, pH modifier and one or more pharmaceutical acceptable excipients, comprising following steps:
1) Co-sifting Upadacitinib with pH modifier and diluent and mixing it to obtain a blend.
2) The blend of step 1 is granulated with required quantity of purified water.
3) The wet granules are wet milled through Co-mill and are dried in Rapid dryer.
4) The dried granules of step 3 are passed through Co-mill with suitable screen.
5) The extragranular materials i.e., release control polymer and glidant are co-sifted and blended with sized granules.
6) Lubricant is added and lubricated the blend of step 5.
7) The Lubricated blend of step 6 is compressed into tablets using compression machine with suitable punches.
8) The core tablets are coated with coating agent using auto coater.

7. The pharmaceutical composition of upadacitinib according to claim 1, wherein the release of upadacitinib is not less than 60% when measured at 8 hours in a dissolution test using USP Type 1 apparatus method at a rotation of 100 rpm in 0.05 M Phosphate Buffer pH 6.8 in 900 mL of volume.

8. The pharmaceutical composition of upadacitinib according to claim 1, wherein the said composition does not have more than 1.0% (w/w) of total impurity of upadacitinib, after being stored at 40° C./75% RH and 25° C./60% RH for at least 6 months.

9. A pharmaceutical composition of upadacitinib comprising upadacitinib or its pharmaceutically acceptable salt thereof, release control polymer, pH modifier and one or more pharmaceutical acceptable excipients; wherein the said release control polymer is hydrophobic polymer selected from the group consisting of glyceryl dibehenate, ethyl cellulose and combinations thereof and the said composition is bioequivalent to innovator product Rinvoq® under both fasting and fed conditions.
